(19) United States
(12) Patent Application Publication
(10) Pub. No.: US 2019/0271660 A1
(43) Pub. Date: Sep. 5, 2019

(54) NANOPORE FORMED THROUGH FIN BY SELF-ALIGNMENT

(71) Applicant: IMEC vzw, Leuven (BE)

(72) Inventors: Boon Teik Chan, Leuven (BE); Zheng Tao, Heverlee (BE); Jean-Francois de Marneffe, Bossut-Gottechain (BE); Chang Chen, Heverlee (BE)

(21) Appl. No.: 16/292,139

(22) Filed: Mar. 4, 2019

(30) Foreign Application Priority Data
Mar. 5, 2018 (EP) 18159987

(51) Int. Cl. G01N 27/414; H01L 29/66; H01L 21/306; H01L 21/308; H01L 21/02; H01L 29/06; H01L 29/78; G01N 33/487; C12Q 1/6869

(52) U.S. Cl. CPC G01N 27/4145; H01L 29/66795; H01L 21/30604; H01L 21/3081; C12Q 1/6869; H01L 29/0665; H01L 29/7853; H01L 21/0217; G01N 33/48721; H01L 21/02164

(57) ABSTRACT

The disclosed technology generally relates to a method of forming a nanoscale opening in a semiconductor structure, and more particularly to forming a nanoscale opening that can be used for sensing the presence of polymers, e.g., the individual bases of deoxyribonucleic acid (DNA) or ribonucleic acid (RNA). In one aspect, a method of forming a nanopore in a semiconductor fin includes providing a fin structure comprising a bottom layer and a top layer, pattering the top layer to form a pillar, and laterally embedding the pillar in a filler material. The method additionally includes forming an aperture in the filler material by removing the pillar, and forming the nanopore in the bottom layer by etching through the aperture. In another aspect, a semiconductor fin is fabricated using the method.

### CROSS-REFERENCE TO RELATED APPLICATIONS

This application claims foreign priority to European Patent Application No. EP 18159987.9, filed on Mar. 5, 2018, the content of which is incorporated by reference herein in its entirety.

### BACKGROUND

### Field

The disclosed technology generally relates to a method of forming a nanoscale opening in a semiconductor structure, and more particularly to forming a nanoscale opening that can be used for sensing the presence of polymers, e.g., the individual bases of deoxyribonucleic acid (DNA) or ribonucleic acid (RNA).

### Description of the Related Technology

In some polymer sensing technologies, long chain polymers such as, e.g., DNA may be characterized by using a semiconductor device based on a field-effect transistor (FET), such as, e.g., a metal oxide semiconductor FET (MOSFET), comprising a drain region, a source region and an opening through which the polymer can pass. The voltage potential applied across the drain and source regions creates a gradient that causes the polymer to move through the opening. In case of, e.g., a DNA or RNA strand, the sequence of bases may induce charges, which can form a conducting channel in a semiconductor channel region between the drain and source regions, resulting in a current variation that can be detected by, e.g., a current meter. Each different type of bases A, C, G and T (in case of a DNA strand) may induce a current having a particular magnitude and waveform representative of the particular charge associated with the respective type of bases. Thus, by studying the current variation as the sample passes through the opening, the sequence of bases can be detected.

Some of these technologies rely on an opening having a highly controlled size and shape. Depending on the type of polymer strand to be sensed, the opening is sometimes preferably configured to have a diameter small enough to allow only one stand to pass through the opening at any given time. DNA sequencing, for example, may be performed using an opening having a diameter within a nanoscale range, such as within the range of about 1 nm to about 10 nm. Forming such small openings are technically challenging, not at least in terms of alignment, and therefore a need exists for improved and more accurate methods for forming such semiconductor devices.

### SUMMARY OF CERTAIN INVENTIVE ASPECTS

An objective of at least a part of the disclosed technology is to provide a method of forming a nanoscale sized opening, also referred to herein as nanopore, in a semiconductor device. A further objective is to provide a method that enables a nanopore to be formed in a semiconductor fin structure, and preferably a fin having a width of 10 nm or less. Further and alternative objectives may be understood from the following.

According to a first aspect of the disclosed technology is a method of forming a nanopore in a semiconductor fin, comprising:

providing a fin structure comprising at least a bottom layer and a top layer,

patterning the top layer to form a pillar,

laterally embedding the pillar in a filler material,

forming an aperture in the filler material by removing the pillar, and

forming the nanopore in the bottom layer by etching though the aperture.

The method makes use of a multiple patterning process that enables a nanopore to be correctly aligned and formed on a fin having a width of the same order as the nanopore, such as about 10 nm or less. In the multiple patterning process, the position of the nanopore is aligned in a width direction, or lateral direction of the fin in a first step and aligned in a length direction of the fin in another step. The disclosed technology is based on the realization that by employing a pillar structure formed of a top layer and a bottom layer, and cutting the top layer into a pillar structure, the pillar structure can be used as a sacrificial structure for forming an etch aperture that is self-aligned in the lateral direction of the fin. The present process allows for an improved definition and positioning of the nanopore compared to etching the nanopore in a single lithographic step. Thus, by utilizing self-alignment to position the etch mask in the lateral direction of the fin, a semiconductor structure resembling a fin field-effect transistor (finFET) is enabled, which is small enough to be used for sensing polymers such as, e.g., DNA or RNA strands. In such a device, an opening or pore having a width of about 1 nm to about 10 nm may be arranged between the drain region and the source region of the fin to form a channel as the sample moves through the opening. The disclosed technology is advantageous in that it allows for the lateral alignment of the nanopore to be improved, thereby allowing for a reduced ratio between the fin width and the nanopore diameter. A reduced fin width, or a reduced cross-section of the fin, is advantageous in that it may reduce the drive current during operation of the device.

As used herein, a pillar refers to a structure extending in vertical direction, e.g., a direction normal to a major surface of the substrate. The pillar may also be referred to as a dot or mask feature that can be used as a sacrificial structure for forming the aperture through which the nanopore is etched.

As used herein, the term vertical (for instance with reference to a direction or a plane or the pillar) denotes a geometrical axis being parallel to a stacking direction of the layers of the fin structure, e.g., a direction normal to a major surface to the substrate. Correspondingly, a vertical axis may be perpendicular to a main plane of extension or a main surface of the substrate or any of coplanar layers formed thereon, such as the bottom layer or the top layer. Terms such as above and under as used herein may accordingly refer to opposite directions along the vertical axis, with respect to a reference. As herein, the term horizontal denotes a horizontal axis being perpendicular to the vertical axis.

The device resulting from the method may include a substrate supporting the afore-mentioned layers forming the fin structure. In this case, a ‘vertical’ direction/plane may be understood as a direction/plane being perpendicular to a main plane of extension or a main surface of the substrate. Correspondingly, a ‘horizontal’ direction/plane may be understood as a direction parallel to a main plane of extension or a main surface of the substrate.

The terms fin or fin-structure as used herein may refer to a fin-shaped feature having a length and width extension in the horizontal direction and a height extension in the vertical direction. The width direction may also be referred to as a lateral direction of the fin. The fin-shaped features may be formed using standard fin-based processes, in which for example the stacked structure of the bottom layer and the top layer may be provided with trenches defining and separating the fins.

The term nanopore as used herein refers to an opening or channel extending in, and preferably through, the fin. The length extension of the opening may be oriented in the vertical direction. The prefix nano refer to the fact that a diameter, or width or cross sectional size of the pore may lie within the range of about 1 nanometer to about 10 nanometer. The nanopore may have a square-shaped or circular cross section, or anything in between. Further examples will be discussed in the following in connection with some of the embodiments.

According to an embodiment, a line mask may be used to form the top layer of the fin structure into the pillar. The line mask may extend in a direction intersecting the length direction of the fin, such as for example orthogonal to the length direction of the fin, such that the region in which the line mask overlaps the material of the top layer defines the etch mask used when cutting the top layer into the pillar. The resulting pillar may thus be self-aligned in the lateral direction on the fin, whereas the lengthwise position may be determined by the lithographic process forming the line mask.

According to an embodiment, the step of forming the aperture in the filler material may further include lining the aperture with a spacer material, thereby reducing a size of the aperture. The spacer material may, for example, be provided in an atomic layer deposition (ALD) process or by oxidation of the sidewalls of the aperture. Adding the spacer material allows for the size of the resulting nanopore to be reduced to a desired size, such as down to a diameter as small as 1 nm. The spacer material may, for example, be silicon dioxide (SiO₂), silicon nitride (Si₃N₄), silicon oxycarbide (SiOC) or a metal such as titanium nitride (TiN), tantalum nitride (TaN) or aluminum nitride (AlN).

According to an embodiment, the top layer may comprise a first mask material and a second mask material arranged on top of the first mask material, wherein the first material is an etch stop material protecting the bottom layer during etching of the second mask material. This allows for a better process control of the top layer patterning, resulting in a reduced risk of etch-back of the bottom layer during the pillar formation. Hence, a method is obtained which is compatible with a bottom layer having a reduced thickness.

According to an embodiment, the first mask material may be formed of SiO₂, Si₃N₄, SiOC, and silicon oxynitride (SiON). Depending on the type of material selected for the first mask material, the second mask material may be formed of amorphous silicon (a-Si), TiN, SiO₂, Si₃N₄, SiOC, and silicon oxynitride (SiON). Preferably, the first mask material and the second mask material are selected so as to achieve an etch selectivity between the mask layers, thereby enabling an etch stopping effect of the double mask. Such combinations may for example be a first mask material of SiO₂ combined with a second mask material of a-Si. This is particularly advantageous when the bottom layer, forming the fin, is formed of Si, since the intermediate first mask material of SiO₂ allows for the pillar pattern to be transferred into the second mask material without damaging or etching back the underlying Si fin.

The filler material may be selected such that an etch selectivity to the pillar material is achieved. The filler material may for example be SiO₂ or Si₃N₄.

The bottom layer, of which the resulting fin may be formed, may for example be formed of silicon, such as the top silicon layer of a silicon on insulation (SOI) substrate.

The nanopore may in one example have a substantially uniform diameter, or cross section, along its length. However, other configurations are also possible. In an embodiment, the nanopore may have a tapered or funnel-shaped profile, e.g., a diameter that decreases towards the bottom or base of the fin. Such a gradually reduced opening size may allow for an improved control of the flow through the opening, preferably such that only one sample strand at the time is guided through the opening. The tapered profile may, for example, be obtained by a wet etching process, such as potassium hydroxide (KOH) etching, resulting in a V-shaped profile along the (111) planes (in case of Si). Such a profile may comprise a facet of 54.7° to the silicon surface. Other examples include reactive ion etching (RIE), which may be sequenced with sidewall passivation, using for example polymerization, to achieve the desired cross sectional profile of the opening.

It will be appreciated that the forming of the nanopore may further comprise material deposition steps in order to yield even smaller openings. This may include thermal oxidation or anodic oxidation, which has the advantage of enabling the size of the opening to be monitored during the oxidation, and ALD deposition similar to the processed discussed above. Thus, according to an embodiment the step of forming the nanopore in the bottom layer may comprise the additional step of lining the nanopore with a spacer material so as to further reduce the size of the nanopore.

According to a second aspect, a semiconductor fin comprising a nanopore generated by using a method according to the first aspect is disclosed.

According to a third aspect, a transistor structure for DNA sensing is disclosed, wherein the transistor structure comprises a semiconductor fin according to the second aspect.

The devices according to the second and third aspects may generally present the same or corresponding advantages as the method according to the first aspect and the embodiments associated therewith. Thus, it is appreciated that the embodiments discussed in connection with the first aspect are equally combinable with the devices according to the second and third aspects, and are therefore not repeated in the present disclosure.

### BRIEF DESCRIPTION OF THE DRAWINGS

The above, as well as additional objects, features and advantages of the disclosed technology, will be better understood through the following illustrative and non-limiting detailed description, with reference to the appended drawings. In the drawings like reference numerals will be used for like elements unless stated otherwise.

FIGS. 1A-1E illustrate intermediate structures at various stages of a method of forming a nanopore in a semiconductor fin, according to embodiments.

FIGS. 2A-2E illustrate intermediate structures at various stages of a variation of a method of forming a nanopore in a semiconductor fin, according to embodiments.

FIG. 3 illustrate a device comprising a semiconductor fin and a nanopore, according to embodiments.

FIG. 4 is a flowchart schematically illustrating a method of forming a nanopore in a semiconductor fin, according to embodiments.

### DETAILED DESCRIPTION OF CERTAIN ILLUSTRATIVE EMBODIMENTS

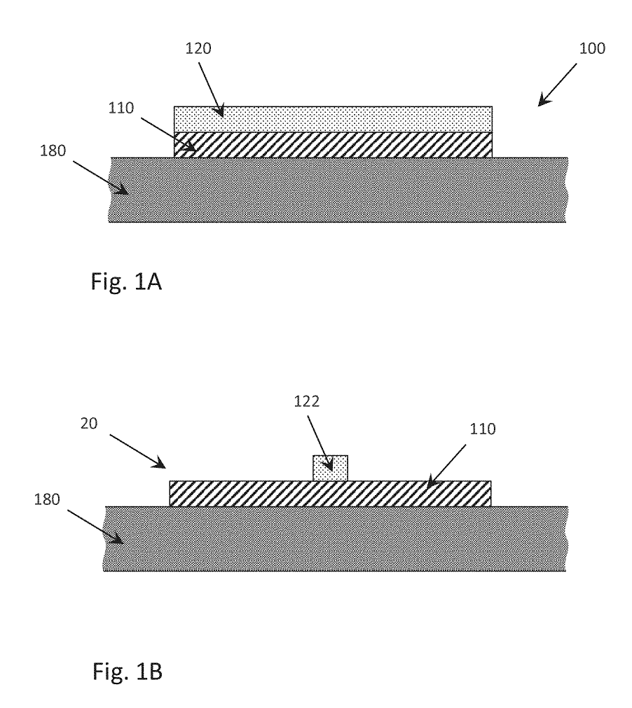

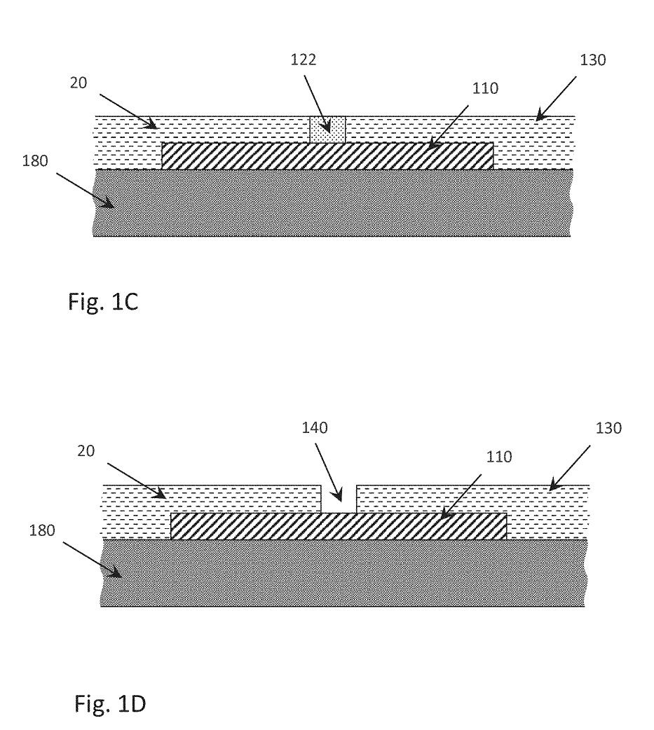

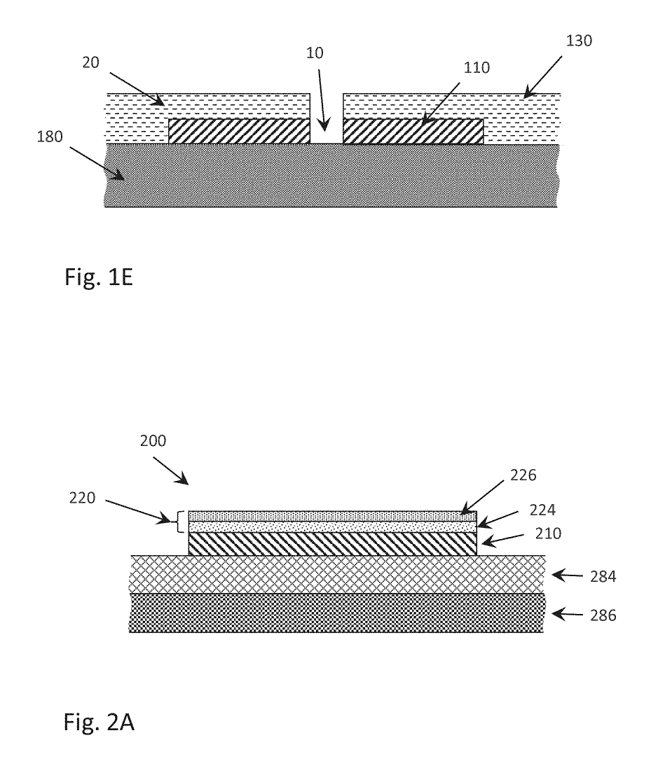

A method of forming a nanopore in a semiconductor fin will now be described with reference to FIGS. 1A-1E, illustrating intermediate structures at various stages of forming a nanopore in a fin structure according to embodiments, using vertical cross sections taken along the length direction of the fin structure.

FIG. 1A shows a fin structure 100 comprising a bottom layer 110 and a top layer 120 arranged on a substrate 180. The bottom layer 110 may, for example, be formed of Si, such as of a SOI wafer, and the top layer 120 of, for example, SiO₂, Si₃N₄ or other materials which, preferably, provides an etch selectivity relative the material of the bottom layer 110. The bottom layer 110 and the top layer 120 may be provided in the form of a stack, which may be patterned into one or several fin structures 100 using a suitable fin patterning process, which may include a lithography process and an etch process.

In a subsequent step, the top layer 120 may be patterned into a pillar structure 122 as shown in the intermediate structure illustrated in FIG. 1B. This may, for example, be achieved by forming a line mask (not shown) over the fin structure 100 (FIG. 1A) across the fin structure 100 and a following etch process, leaving a pillar or dot shaped mask structure 122 on top of the fin 20 formed by the bottom layer 110. The position of the line mask in the length direction of the fin structure 100 determines the alignment of the pillar structure 122 along the fin 20, whereas the pillar structure 122 is self-aligned in the lateral direction (e.g., the width direction) of the fin 20.

In the intermediate structure illustrated in FIG. 1C, the pillar 122 has been embedded in a filler layer 130, which, for example, may be formed of a dielectric filling material such as Si₃N₄ of SiO₃. In the present example, the top surface of the structure has been planarized, for example by means of a chemical-mechanical planarization (CMP) process.

FIG. 1D shows an intermediate structure after the pillar 122 has been removed, leaving an aperture 140 that can be used as an etch mask for the resulting nanopore 10. The pillar 122 may hence be referred to as a sacrificial pillar 122. The material of the pillar 122 may, for example, be etched in a suitable wet etch process, such as tetramethylammonium hydroxide (TMAH) in case the pillar 122 is formed of, for example, amorphous Si. Advantageously, the TMAH etch is used in combination with an etch stop layer separating the pillar 122 and the underlying fin material.

The pattern defined by the aperture 140 in the filler material layer 130 may then be transferred into the fin 20 to form the nanopore 10. An example of the resulting device is illustrated in FIG. 1E, depicting the semiconductor fin 20 when provided with the nanopore 10 extending in a top-bottom direction of the fin 20. The nanopore 10 is self-aligned in the width direction of the fin 20. The self-alignment is achieved due to the use of the sacrificial pillar 122 which is formed of the top layer 120 of the fin structure 100. The resulting fin 20 has a filler material formed thereover, and the nanopore 10 is formed in a vertical direction through the filler layer 130 and further through the semiconductor fin, such that the nanopore 10 exposes a surface of the underlying substrate 180. The illustration in FIG. 1E may also represent a final device.

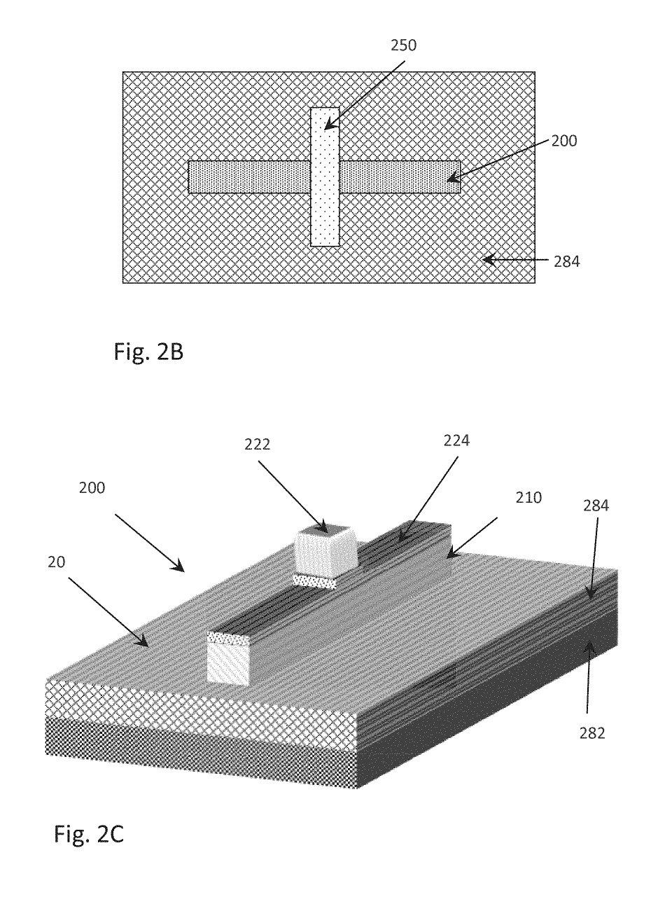

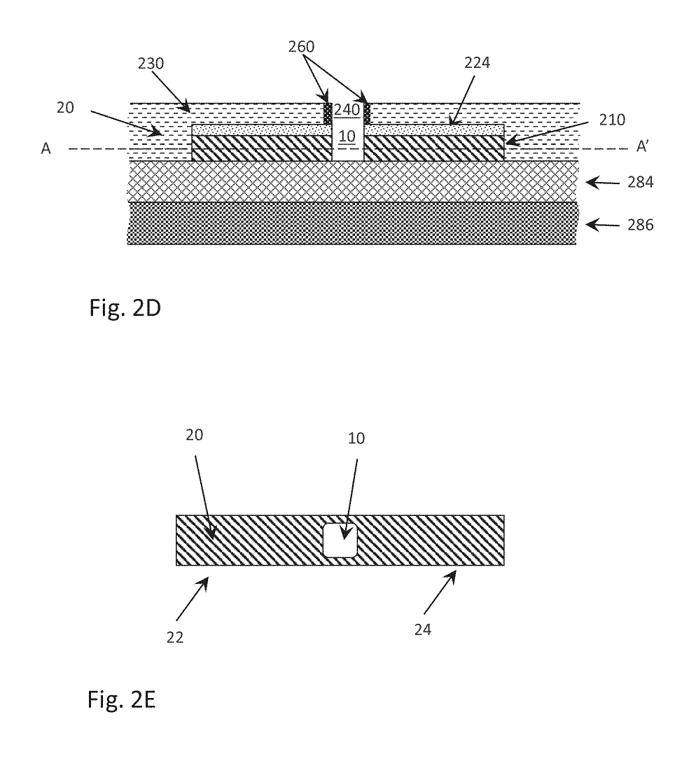

The above-described example method of forming the nanopore may be varied in several ways, using different material combinations, material layers and processing steps. An example of such variation will now be discussed with reference to FIGS. 2A-2E. It will be appreciated that the depicted examples may be similarly configured as the embodiments described above in connection with FIGS. 1A-1E.

FIG. 2A shows a cross-sectional side view of an intermediate structure including a fin structure 200. The fin structure 200 comprises a bottom layer 210 formed of. for example Si, arranged on a substrate comprising a dielectric layer 284, such as for example SiO₂, and a Si layer 286. The bottom layer 210, the dielectric layer 286 and the Si layer 282 may in some examples be formed from a silicon-on-insulator (SOI) wafer.

The fin structure 200 may further comprise a first mask material 224 and a second mask material 226, which together may form the top layer 220 of the fin structure 200. The first mask material 224 may be arranged between the bottom layer 210 and the second mask material 226 so as to form an interface between the top layer 220 and the bottom layer 210. In this way, the top layer 210 may be referred to as a dual mask. The first mask material 224 may in some examples be or comprise a dielectric such as SiO₂, Si₃N₄, SiOC or SiON. The second mask material 226 may be or comprises amorphous Si, TiN, Si₃N₄, SiO₂, SiOC or SiON, depending on the type of material used in the first mask 224. Preferably, the first and second mask materials 224, 226 are selected such that the combination allows for an etch selectivity between the materials, which advantageously allows for the first mask material 224 to serve as an etch stop layer during the formation of the pillar 222. In the present example, the first mask material is or comprises SiO₂ and the second mask material is or comprises amorphous Si.

The the pillar 222 (FIG. 2C) is formed using the top layer, and in this example the second mask material 226, to be patterned into the desired structure. The patterning may, for example, be performed by means of a lithography and etching. For this purpose, a hard mask formed of, for example, spin-on-carbon (SoC) and spin-on-glass (SoG) may be used (not shown). Alternatively, a dielectric anti-reflective coating (DARC, e.g., SiOC) may be provided on an advanced patterning film (APF, e.g., a carbon film) stack. Photoresist may be employed to pattern the SoC and SoG layers into a line mask 250 that can be used when cutting the second mask material 226 of the fin structure 200 into a self-aligned pillar or dot structure, using the first mask material 224 as an etch stop.

FIG. 2B shows a top view of an intermediate structure including the fin structure 200 of FIG. 2A, in which the line mask 250 crosses the fin structure 200 in a substantially orthogonal manner.

A perspective view an intermediate structure including the fin structure 200 is shown in FIG. 2C. The fin structure 200 comprises a fin 20 formed of the bottom layer 210, a pillar 222 arranged on top of the fin 20, and the etch stop layer 224 arranged in between. FIG. 2C indicates that the etch stop layer 224 has been slightly etched back during the forming of the pillar 222. It will be appreciated that in some implementations, the pillar 222 may be formed without the intermediate etch stop layer 224, e.g., formed from a stack comprising the bottom layer 210 and the top layer (e.g., the second mask material 226 or the top layer 120 (FIG. 1A)). In that case, it might be advantageous to use a slightly thicker bottom layer so as to compensate for possible etch back effects.

FIG. 2D is a cross sectional side view of an intermediate structure or a final device including the fin shown in FIG. 2C, after the pillar 222 has been embedded in the filler material 230 and replaced by the aperture 240. As also shown in FIG. 2D, the aperture 240 may be lined with a spacer material 260 provided by for example oxidation or ALD. The spacer material 260 may be added so as to reduce the diameter of the aperture 260 and thereby enable a smaller nanopore 10 to be formed in the fin 20. Even though not shown in the present cross section, the spacer material 260 may be provided on all sidewalls of the aperture 240, which hence may have a width or diameter that is smaller than the width of the fin 20. This allows for the nanopore 10 to be etched through the fin material without breaking through the sidewalls of the fin 20. The spacer material 260 may for example be a dielectric such as SiO₂, Si₃N₄, SiOC, TiO₂, ZrO₂ or HfO₂ or a metal such as TiN, TaN or AlN.

FIG. 2E is a cross sectional top view of an intermediate structure or a final device including the fin 20 shown in FIG. 2D, taken along the section A-A′. As shown in FIG. 2E, the nanopore may be centrally located in the width direction of the fin 20, allowing a polymer such as, e.g., a DNA or RNA strand to pass through the fin and thereby form a channel between the drain region 22 and the source region 24 of the fin 20. In the present example, the fin 20 may have a width of about 10 nm and a height of about 10 nm to about 20 nm. The nanopore 10 may in the present example have a width of about 5 nm. Thus, in the illustrated embodiment, a lateral dimension of the nanopore in a width direction of the semiconductor fin is smaller than the width of the fin, such that the nanopore is laterally confined within the semiconductor fin. The resulting nanopore 10 in the illustrated embodiment is arranged such that a portion of the nanopore formed through the filler material 230 is lined with a spacer material 260, while the spacer 260 material does not extend into a portion of the nanopore 10 formed through the fin 20.

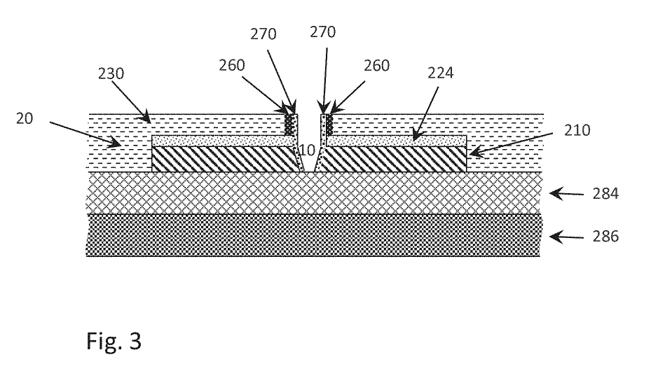

FIG. 3 illustrates a semiconductor fin 20 according to another example. The semiconductor fin 20 may be similarly configured as the previously described embodiments, but differ in that the nanopore 10 is provided with a tapered or funnel shaped profile. As shown in FIG. 3, the opening 10 may decrease towards the base of the fin 20. This tapered profile may for example be achieved by a reactive ion etch (RIE) process, in which etching is cycled with sidewall passivation. Alternatively, or additionally an anisotropic wet etch, such as, e.g., orientation dependent potassium hydroxide (KOH) etch of Si, may be employed to provide a V-shaped profile of the opening 10.

Further, the nanopore 10 may be provided with a spacer material 270 lining, e.g., conformally lining, the sidewalls of the nanopore 10. The spacer material 270 may be similar to the one used for the aperture 140, and allows for the diameter of the pore 10 to be even further decreased.

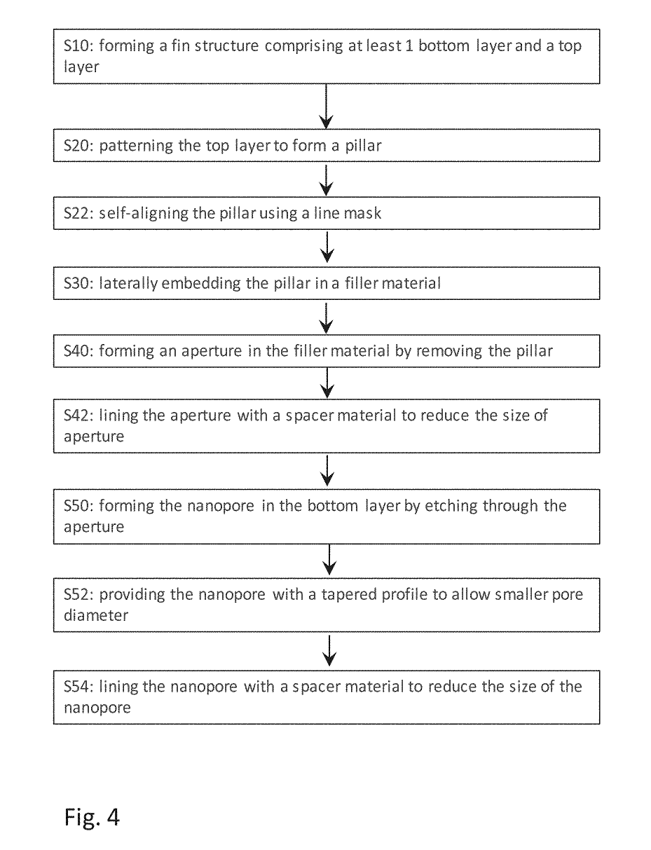

FIG. 4 is a flowchart illustrating a method according to embodiments described above. The method may comprise the steps of providing S10 a fin structure comprising at least a bottom layer and a top layer, patterning S20 the top layer to form a pillar, laterally embedding S30 the pillar in a filler material, forming S40 an aperture in the filler material by removing the pillar, and forming S50 the nanopore in the bottom layer by etching through the aperture. The patterning S20 the top layer to form a pillar may include a self-aligning S22 of the pillar using a line mask. Further, the aperture may be lied S42 with a spacer material to reduce the size of the aperture and hence the nanopore. This also applies to the nanopore itself, which may be lined S54 with the same or a similar material. In some embodiments, the nanopore additionally, or alternatively, may be provided with a tapered S52 profile so as to allow for an even smaller pore diameter to be achieved.

In the above the inventive concept has mainly been described with reference to a limited number of examples. However, as is readily appreciated by a person skilled in the art, other examples than the ones disclosed above are equally possible within the scope of the inventive concept, as defined by the appended claims.

What is claimed is:

1. A method of forming a nanopore, the method comprising:
providing a fin structure comprising a bottom layer and a top layer;
pattering the top layer to form a pillar;
laterally embedding the pillar in a filler material;
forming an aperture in the filler material by removing the pillar; and
forming the nanopore in the bottom layer by etching through the aperture.

2. The method of claim 1, wherein patterning the top layer comprises self-aligning the pillar on the bottom layer using a line mask intersecting the fin structure.

3. The method of claim 1, wherein forming the aperture further comprises lining the aperture with a spacer material, thereby reducing a size of the aperture.

4. The method of claim 1, wherein the top layer comprises a first mask material and a second mask material arranged on top of the first mask material, and wherein the first mask material serves as an etch stop material protecting the bottom layer during etching of the second mask material.

5. The method of claim 4, wherein the first mask material is selected from the group consisting of silicon dioxide (SiO₂), silicon nitride (Si₃N₄), silicon oxycarbide (SiOC) and silicon oxynitride (SiON), and wherein the second mask material is selected from the group consisting of amorphous silicon (a-Si), titanium nitride (TiN), silicon dioxide (SiO₂), silicon nitride (Si₃N₄), silicon oxycarbide (SiOC) and silicon oxynitride (SiON).

6. The method of claim 1, wherein the filler material is selected from the group consisting of silicon dioxide (SiO₂) and silicon nitride (Si₃N₄).

7. The method of claim 1, wherein the spacer material is selected from the group consisting of silicon dioxide (SiO₂), silicon nitride (Si₃N₄), silicon oxycarbide (SiOC) and a metal.

8. The method of claim 1, wherein forming the nanopore comprises forming a tapered nanopore.

9. The method of claim 1, wherein the bottom layer comprises silicon.

10. The method of claim 1, wherein forming the nanopore comprises lining the nanopore with a spacer material, thereby reducing a size of the nanopore.

11. A semiconductor structure with at least one nanopore, comprising:
a semiconductor fin extending in a lateral length direction over a substrate;
a dielectric filler material formed over the semiconductor fin; and
a nanopore formed in a vertical direction through the filler material and further through the semiconductor fin, such that the nanoscale pore exposes a top surface of the substrate.

12. The semiconductor structure of claim 11, wherein the semiconductor fin has a width less than about 10 nm.

13. The semiconductor structure of claim 12, wherein a lateral dimension of the nanopore in a lateral width direction of the semiconductor fin is smaller than the width of the fin such that the nanopore is laterally confined within the semiconductor fin.

14. The semiconductor structure of claim 13, wherein a portion of the nanopore formed through the dielectric filler material is lined with a spacer material different from the dielectric filler material, wherein the spacer material does not extend into a portion of the nanopore formed through the semiconductor fin.

15. The semiconductor structure of claim 14, further comprising an etch stop layer vertically interposed between the semiconductor fin and the dielectric filler material, wherein the etch stop layer is formed of a material different from the dielectric filler material and the spacer material.

16. A transistor device with at least one nanopore, comprising:
a semiconductor fin extending in a lateral length direction over on a substrate;
a dielectric filler material formed over the semiconductor fin;
a nanopore formed in a vertical direction through the filler material and further through the semiconductor fin, such that the nanoscale pore exposes a top surface of the substrate; and
a source and a drain on formed on opposite sides of the nanopore in the semiconductor fin, such that the transistor device is configured for DNA sensing.

17. The semiconductor structure of claim 16, wherein a lateral dimension of the nanopore in a lateral width direction of the semiconductor fin is smaller than the width of the fin such that the nanopore is laterally confined within the semiconductor fin.
